(19) United States
(12) Patent Application Publication
(10) Pub. No.: US 2024/0325653 A1
(43) Pub. Date: Oct. 3, 2024

(54) DUAL PIEZOELECTRIC STACK PLUNGER SUB-ASSEMBLY

(71) Applicant: BECTON, DICKINSON AND COMPANY, Franklin Lakes, NJ (US)

(72) Inventor: Alessandro E. PIZZOCHERO, Chelmsford, MA (US)

(73) Assignee: Becton, Dickinson and Company, Franklin Lakes, NJ (US)

(21) Appl. No.: 18/283,626

(22) PCT Filed: Mar. 23, 2022
(86) PCT No.: PCT/US2022/021542

§ 371 Date: Sep. 22, 2023

Related U.S. Application Data
(60) Provisional application No. 63/165,642, filed on Mar. 24, 2021.

(51) Int. Cl. A61M 5/315

(52) U.S. Cl. CPC A61M 5/31511; A61M 2005/31518; A61M 2005/31588; A61M 2205/0294

(57) ABSTRACT

A piezoelectric actuator is provided for use within the barrel of a liquid medication delivery syringe. The actuator includes a plunger, a frame attached to the plunger, an advance piezoelectric unit disposed within the frame, and a lock piezoelectric unit disposed within the frame. Each of the piezoelectric units includes a number of piezoelectric elements arranged such that application of an electric field to piezoelectric elements of the lock unit causes the elements to expand in a radial direction of the barrel, locking the frame in position within the barrel. Application of an electric field to piezoelectric elements of the advance unit cause the elements to expand in an axial direction of the barrel, pumping liquid medication, disposed within the syringe, out of the barrel.

This application claims the benefit of U.S. Provisional Application No. 63/165,642, filed Mar. 24, 2021 and PCT International Application No. PCT/US2022/021542, filed Mar. 23, 2022, the entire contents of which are hereby incorporated by reference herein.

### BACKGROUND

### 1. Field

Apparatuses and methods consistent with example embodiments relate to a plunger sub-assembly for delivery of a liquid pharmaceutical product, and more particularly, to a plunger sub-assembly comprising dual piezoelectric stacks.

### 2. Description of the Related Art

Diabetes is a group of diseases characterized by high levels of blood glucose resulting from the inability of diabetic patients to maintain proper levels of insulin production when required. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. An effective method for insulin therapy and managing diabetes is infusion therapy or infusion pump therapy in which an insulin pump is used. An insulin delivery device (IDD) may include an insulin pump that can provide continuous infusion of insulin to a diabetic patient at varying rates in order to more closely match the functions and behavior of a properly operating pancreas of a non-diabetic person that produces the required insulin, and the insulin pump can help the diabetic patient maintain his/her blood glucose level within target ranges based on the diabetic patient's individual needs. Infusion pump therapy requires an infusion cannula, typically in the form of an infusion needle or a flexible catheter, that pierces the diabetic patient's skin and through which infusion of insulin takes place. Infusion pump therapy offers the advantages of continuous infusion of insulin, precision dosing, and programmable delivery schedules.

Currently, there are two principal modes of daily insulin therapy for the treatment of type 1 diabetes. The first mode includes syringes and insulin pens that require a needle stick at each injection, typically three to four times per day that are simple to use and relatively low in cost. Another widely adopted and effective method of treatment for managing diabetes is the use of an insulin pump. Insulin pumps can help the user keep blood glucose levels within target ranges based on individual needs, by continuous infusion of insulin. By using an insulin pump, the user can match insulin therapy to lifestyle, rather than matching lifestyle to how an insulin injection is working for the user.

Conventional insulin pumps are capable of delivering rapid or short-acting insulin 24 hours a day through a catheter placed under the skin. Insulin doses are typically administered at a basal rate and in a bolus dose. Basal insulin is delivered continuously over 24 hours, and keeps the user's blood glucose levels in a consistent range between meals and overnight. Some insulin pumps are capable of programming the basal rate of insulin to vary according to the different times of the day and night. Bolus doses are typically administered when the user takes a meal, and generally provide a single additional insulin injection to balance the carbohydrates consumed. Some conventional insulin pumps enable the user to program the volume of the bolus dose in accordance with the size or type of the meal consumed. Conventional insulin pumps also enable a user to take in a correctional or supplemental bolus of insulin to compensate for a low blood glucose level at the time the user is calculating a meal bolus.

There are many advantages of conventional insulin pumps over other methods of diabetes treatment. Insulin pumps deliver insulin over time rather than in single injections and thus typically result in less variation within the blood glucose range that is recommended by the American Diabetes Association. Conventional insulin pumps also reduce the number of needle sticks which the patient must endure, and make diabetes management easier and more effective for the user, thus considerably enhancing the quality of the user's life.

A major disadvantage of existing insulin pumps is that, in spite of their portability, they include multiple components and can be heavy and cumbersome to use. They are also typically more expensive than other methods of treatment. From a lifestyle standpoint, the conventional pump with its associated tubing and infusion set can be inconvenient and bothersome for the user.

Unlike a conventional infusion pump, a patch pump is an integrated device that combines most or all of the fluidic components, including the fluid reservoir, pumping mechanism and a mechanism for automatically inserting the cannula, in a single housing which is adhesively attached to an infusion site on the patient's skin, and does not require the use of a separate infusion or tubing set. Some patch pumps wirelessly communicate with a separate controller (as in one device sold by Insulet Corporation under the brand name OmniPod®), while others are completely self-contained. Such devices are replaced on a frequent basis, such as every three days, when the insulin supply is exhausted.

As a patch pump is designed to be a self-contained unit that is worn by the diabetic patient, it is preferable to be as small as possible so that it does not interfere with the activities of the user. In order to minimize discomfort to the user, it is preferable to minimize the overall dimension of the patch pump. However, in order to minimize the overall dimensions of the patch pump, its constituent parts should be reduced in size as much as possible.

With respect to a patch pump or other insulin delivery device (IDD), the pump may be actuated by a syringe actuator. A conventional syringe includes a plunger attached to a stem which must be at least as long as a length of the syringe barrel. This unfortunately adds to the required size of the patch pump or other IDD.

### SUMMARY

Example embodiments may address at least the above problems and/or disadvantages and other disadvantages not described above. Also, example embodiments are not required to overcome the disadvantages described above, and may not overcome any of the problems described above.

According to an aspect of an example embodiment, a piezoelectric comprises: a plunger, a frame attached to the plunger, an advance piezoelectric unit disposed within the frame, the advance piezoelectric unit comprising a first piezoelectric element, wherein application of an electric field to the first piezoelectric element causes the first piezoelectric element to expand along a first axis, and a lock piezoelectric unit disposed within the frame, the lock piezoelectric unit comprising a second piezoelectric element, wherein application of an electric field to the second piezoelectric element causes the second piezoelectric element to expand along a second axis, orthogonal to the first axis. Alternately, the second axis may form an acute angle with respect to the first axis.

The plunger may have an exterior circumference configured to fit within a barrel.

The first piezoelectric element may comprise a plurality of first piezoelectric elements arranged in a stack.

The second piezoelectric element may comprise a plurality of second piezoelectric elements arranged in a stack.

According to an aspect of another example embodiment, a fluid medication delivery method comprises: holding a fluid medication within a barrel of a syringe, wherein the syringe comprises the barrel and a piezoelectric actuator within a barrel of a syringe, the piezoelectric actuator comprising a plunger, a frame attached to the plunger, an advance piezoelectric unit disposed within the frame and comprising a first piezoelectric element, and a lock piezoelectric unit disposed within the frame and comprising a second piezoelectric element; applying a first electric field to the second piezoelectric element thereby causing the second piezoelectric element to expand in a radial direction of the barrel and exert a pressure on an inner circumference of the barrel, clamping the frame in a rear position in the barrel; while maintaining the first electric field applied to the second piezoelectric element, applying a second electric field to the first piezoelectric element thereby causing the first piezoelectric element to extend along an axial direction of the barrel, substantially orthogonal to the radial direction, thereby advancing the plunger toward a front end of the barrel and pumping fluid medication out of the syringe; while maintaining the second electric field applied to the first piezoelectric element, removing the first electric field from the second piezoelectric element thereby causing the second piezoelectric element to retract in the radial direction away from the inner circumference of the barrel, releasing the clamping of the frame; and releasing the second electric field from the first piezoelectric element thereby causing the first piezoelectric element to retract in the axial direction pulling the frame toward the plunger.

The operations may be repeated, thereby pumping additional fluid medication out of the syringe.

According to an aspect of another example embodiment, a fluid medication pump comprises: a barrel, and a piezoelectric actuator disposed within the barrel, the piezoelectric actuator comprising: a plunger, a frame attached to the plunger, an advance piezoelectric unit disposed within the frame, the advance piezoelectric unit comprising a first piezoelectric element, wherein application of an electric field to the first piezoelectric element causes the first piezoelectric element to expand along a first axis, and a lock piezoelectric unit disposed within the frame, the lock piezoelectric unit comprising a second piezoelectric element, wherein application of an electric field to the second piezoelectric element causes the second piezoelectric element to expand along a second axis, orthogonal to the first axis. Alternately, the second axis may form an acute angle with respect to the first axis.

### BRIEF DESCRIPTION OF THE DRAWINGS

The above and/or other example aspects and advantages will become apparent and more readily appreciated from the following description of example embodiments, taken in conjunction with the accompanying drawings in which:

FIG. 1 is a perspective view of a dual stack piezoelectric syringe actuator according to an example embodiment;

FIGS. 2A-2E are cross-sectional views of a dual stack piezoelectric syringe actuator, disposed in various positions within a barrel of a syringe, according to an example embodiment;

FIG. 3 is a cross-sectional view of a dual stack piezoelectric syringe actuator according to another example embodiment;

FIG. 4 is a perspective view of the dual stack piezoelectric syringe actuator of FIG. 3;

FIG. 5 is a perspective, cross-sectional view of the dual stack piezoelectric syringe actuator of FIG. 3; and

FIG. 6 is a block diagram of an IDD according to an example embodiment.

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these example embodiments that are obvious to those of ordinary skill in the technical field to which these example embodiments pertain may not be described here in detail.

One or more example embodiments describe a syringe actuator which may be used in a patch pump or other IDD, wherein the syringe actuator includes a plunger attached to a piezoelectric actuation mechanism.

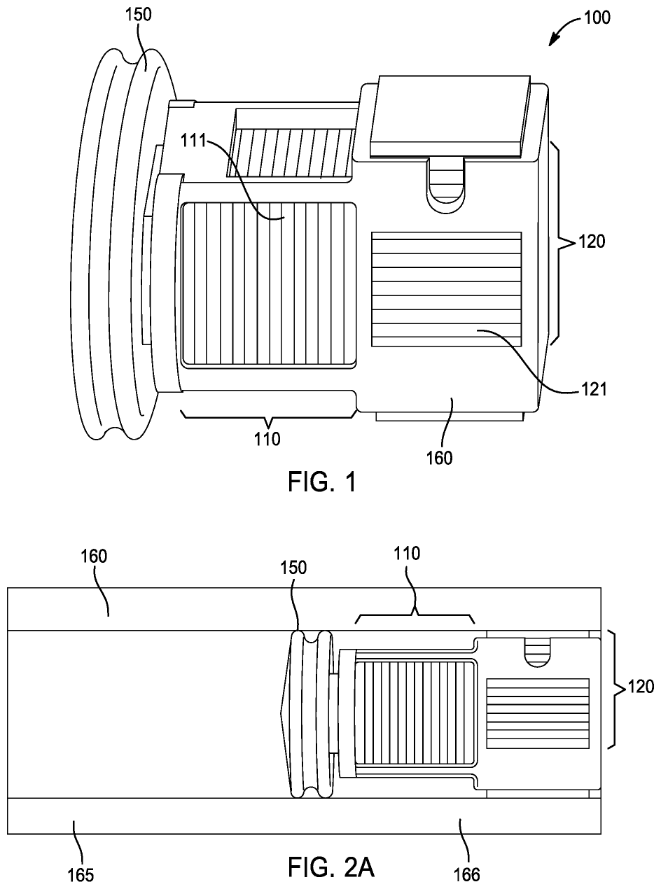

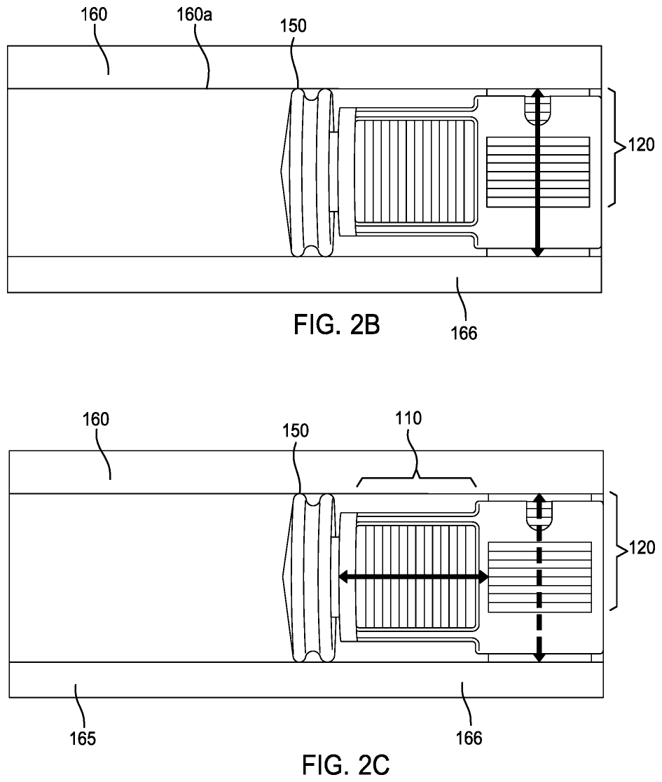

FIG. 1 is a perspective view of a dual stack piezoelectric syringe actuator according to an example embodiment. The actuator 100 is configured to fit within an interior circumference of a barrel of a syringe (see e.g. FIGS. 2A-2E). A cross-section of the barrel may be of any shape, and the actuator 100 includes a plunger 150 having a shape, determined in coordination with the shape of the barrel, to fit snugly within the barrel to thereby seal the barrel, as would be understood by one of skill in the art. For example, a cross-sectional shape of the interior circumference of the barrel, and accordingly the exterior shape of the plunger 150, may be circular, oval, or square with rounded corners as shown, for example, in FIG. 1, or any other shape, as would be understood by one of skill in the art. The plunger 150 may be a single elastomeric piece.

The plunger 150 is attached to a frame 160 holding an advance piezoelectric stack 110 and a lock piezoelectric stack 120. Each of the advance stack 110 and the lock stack 120 may include a plurality of individual piezoelectric elements 111, 121, as shown in FIG. 1. The piezoelectric elements of the advance stack 110 are arranged so that, upon application of an electric field, the elements physically expand in a direction parallel to a length of the barrel. The piezoelectric elements of the lock stack 120 are arranged so that, upon application of an electric field, the elements physically expand in a direction perpendicular to the length of the barrel—in a radial direction of the cross-sectional shape of the barrel. In other words, the piezoelectric elements in the advance stack 110 are arranged orthogonally with respect to the elements of the lock stack 120. Alternately, the piezoelectric elements in the advance stack 110 may be arranged at an acute angle with respect to with respect to the elements of the lock stack 120. As shown in FIG. 1, each stack includes a plurality of piezoelectric elements 111, 121. However, in other example aspects, each stack may include one or more elements. The piezoelectric elements may be made of any piezoelectric material, for example, but not limited to lead zirconate titanate (PZT); barium titanate; lead titanate; ceramic piezoelectric materials, such as gallium nitride or zinc oxide; semiconducting piezoelectric materials; organic polymer piezoelectric materials; and piezoelectric polymer materials.

According to an alternate example aspect, the lock stack, including elements which expand in a radial direction upon application of an electric field, may be replaced with one or more piezoelectric elements, each having opposing outer circumferential surfaces shaped to correspond to an inner circumference of the barrel, wherein each of the one or more piezoelectric elements expands in a radially-outward direction upon application of an electric field. Such elements may each be disc-shaped and may expand radially outward upon application of an electric field, in order to engage with the inner circumference of the barrel.

According to alternate example aspects, the piezoelectric elements may be tube actuators, which are cylinders lined with electrodes that apply an electric field, thus causing radial displacement of the actuators.

According to an example aspect, circumferentially-outer surfaces of the one or more piezoelectric elements of the lock stack may have friction elements, such as rubber feet (not shown), attached thereto, in order to facilitate engagement between the lock stack and the barrel. A viscoelastic material may be disposed between the piezoelectric elements 121 of the lock stack 120 and the inner wall of the barrel 160. Such a material may improve adhesion of the lock stack 120. The viscoelastic material may be provided in a thin layer so motion is not removed via shear effects from the advancing piezo motion. Alternate materials may be selected to be compatible with the material of the barrel 160, its stiffness, and compatibility with the lock stack 120.

Wires and/or flex circuits connect the advance stack and the lock stack to a printed circuit board (PCB) configured to drive the assembly, and the actuator 100 may further include one or more sensors (not shown) to detect motion and/or a position of one or more elements of the actuator 100.

According to an alternate aspect the piezoelectric elements 121 of the lock stack may be spaced apart from the inner walls of the barrel 160 or may be in contact with the inner walls of the barrel 160 when no electric field is applied thereto.

FIGS. 2A-2E are cross-sectional views of a dual stack piezoelectric syringe actuator, disposed in various positions within a barrel of a syringe, according to an example embodiment. FIG. 2A illustrates the actuator 100 disposed in a first, “start” position within a barrel 160 of a syringe. As shown, in the start position, the actuator is located at a rear end 166 of the barrel. A front end 165 of the barrel may be connected to a Y-junction connecting an interior of the barrel to both a fill mechanism and a dispensing port (not shown). A septum may be included between a fill mechanism/reservoir and the barrel, which may be closed off once the barrel has been filled.

FIG. 2B illustrates the actuator 100 in a second, clamping position with the barrel 160. In order to move the actuator 100 from the first position in FIG. 2A to the second position in FIG. 2B, an electric field is applied to the lock stack 120, thereby causing the one or more piezoelectric elements 121 of the lock stack 120 to expand in a radially-outward direction with respect to the barrel 160. As a result of this actuation of the lock stack, in the second, clamping position, the piezoelectric elements 121 of the lock stack 120 are in contact with the inner circumference 160a of the barrel, thereby locking the actuator 100 into its position at the rear end 166 of the barrel CC.

FIG. 2C illustrates the actuator 100 in a third, advancing position within the barrel 160. In this position, while the one or more elements 121 of the lock stack 120 remain actuated, thereby holding the actuator 100 in position at the rear end 166 of the barrel 160, an electric field is also applied to the one or more piezoelectric elements 111 of the advance stack 110, thereby causing the elements 111 to expand along a length of the barrel 160, pushing the plunger 150 toward a front end 165 of the barrel 160, and pumping any liquid medication, such as insulin, out of the barrel.

FIG. 2D illustrates the actuator 100 in a fourth, unclamping position. After the actuation of the advance stack 110 has pushed the plunger forward, as shown in FIG. 2C, the electric field is removed from the lock stack 120, thereby releasing the pressure of the one or more piezoelectric elements 121 on the interior of the barrel 160, as shown in FIG. 2D.

FIG. 2E illustrates the actuator 100 in a fifth, retracting position within the barrel 160. After the lock stack 120 has released its pressure in on the interior of the barrel, as shown in FIG. 2D, the electric field is removed from the advance stack 110, thereby causing the piezoelectric elements 111 of the advance stack 110 to retract and shift the entire actuator 100 forward, toward the front end 165 of the barrel 160. A friction between the plunger 150 and the interior of the barrel prevents the actuator 100 from being pulled back toward the rear end 166 of the barrel 160 when the advance stack 110 retracts.

As would be understood by one of skill in the art, depending on the voltage applied, the dimensions of the barrel, and the properties of the piezoelectric elements 111 of the advance stack 110, the operations advancing the actuator 100 through the positions of FIGS. 2A-2E may need to be repeated in order to deliver a minimum desired dose.

Also, as would be understood by one of skill in the art, the operations illustrated in FIGS. 2A-2E may also be used in varying orders in order to fill the barrel, pulling liquid medication into the barrel from a reservoir.

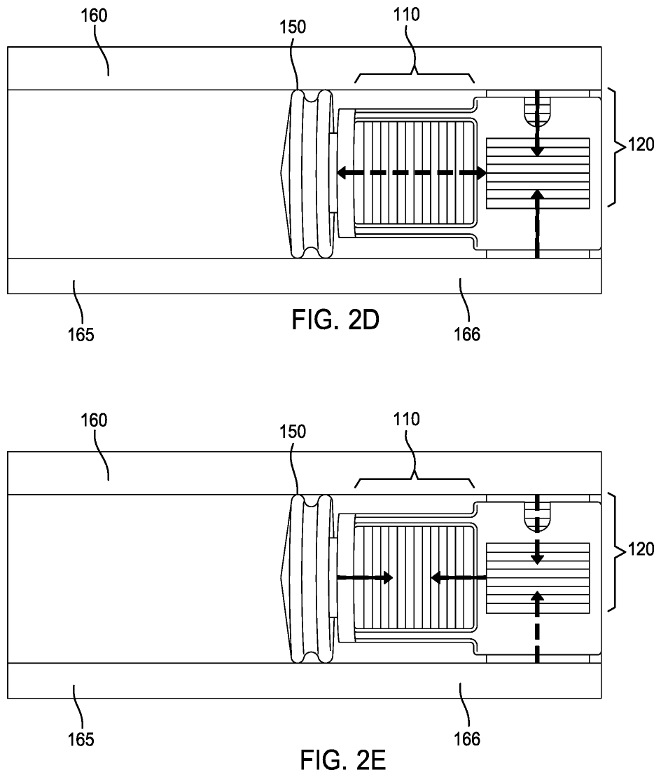

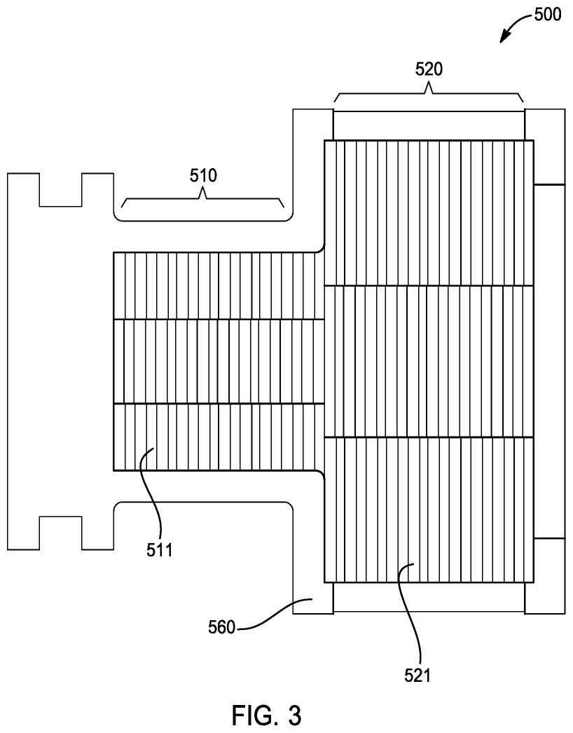

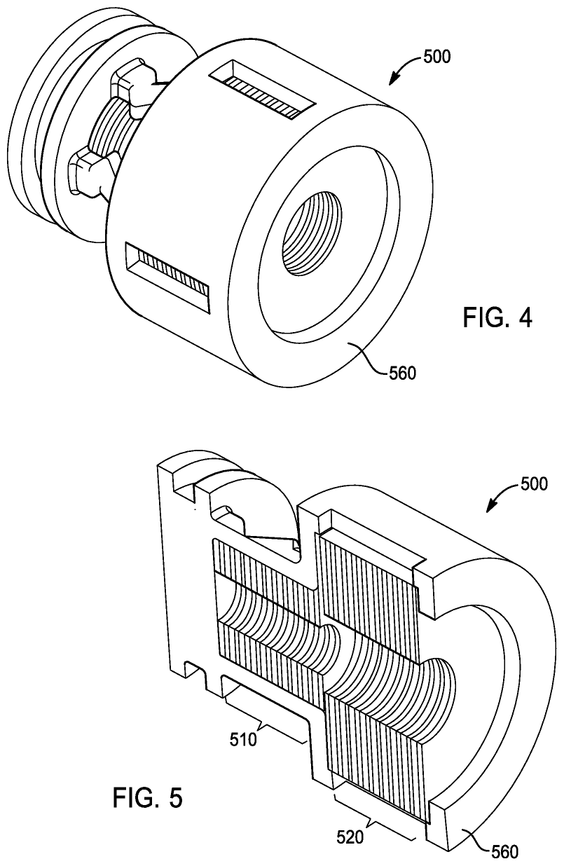

FIGS. 3-5 illustrate a dual stack piezoelectric syringe actuator according to another example embodiment. The actuator 500 is configured to fit within an interior circumference of a barrel of a syringe, for example, a cylindrical barrel.

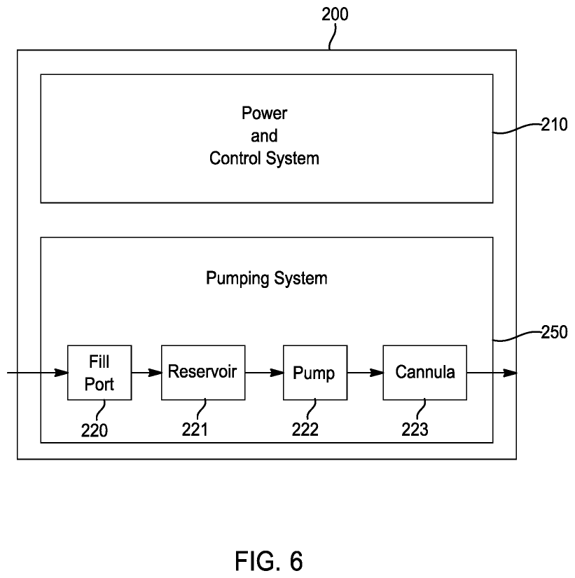

FIG. 6 is a block diagram of an IDD according to an example embodiment. It is to be understood that, although an example actuator is described in conjunction with the example IDD as shown in FIG. 6, this is merely an example, and a dual-stack piezoelectric actuator in accordance with one or more example embodiments may be used in conjunction with any medication delivery system or medical device including a fluid path, as would be understood by one of skill in the art. The actuator 500 includes a frame 560 holding an advance piezoelectric stack 510 and a lock piezoelectric stack 520. Each of the advance stack 510 and the lock stack 520 may include a plurality of individual piezoelectric elements 511, 521. The frame 560 may be made of any of a number of materials and is configured to hold an interior circumference of the barrel of the syringe during an expansion of the lock piezoelectric stack 520, to be flexible enough to allow for the expansion of the advance stack 510, and to be flexible enough to pull the lock piezoelectric stack 520 when it is retracted. The frame 560 holds the lock piezoelectric stack 520 therewithin. This can be achieved by overmolding the frame 560 onto the stacks, swaging a rear portion of the frame 560 to support the lock piezoelectric stack 520, or attaching a separate piece to hold the lock piezoelectric stack 520 to the read of the frame 560. The stacks 510 and 520 may be inserted into the frame 560 from a rear prior to sealing the stacks 510 and 520 within the frame 560.

The individual piezoelectric elements 521 of the lock piezoelectric stack 520 may be disc shaped and may expand in a radially-outward direction upon application of an electric field, in order to engage with the inner circumference of the barrel. The individual piezoelectric elements 511 of the advance piezoelectric stack 510 may expand in a direction parallel to the length of the barrel.

The IDD 200 is an example of a medical device configured for continuous subcutaneous delivery of insulin at set and variable basal (24-hour period) rates and bolus (on-demand) doses for the management of patients with type 2 diabetes mellitus requiring insulin therapy. The IDD 200 includes a power and control system 210, and a pumping system 250. The power and control system 210 may include one or more batteries for providing power for the IDD 200, a microcontroller, a memory, and additional electronics for control and regulation of the pumping system 250, as would be understood by one of skill in the art.

The pumping system 250 includes a reservoir 221 for storing a fluid medication (e.g. insulin) to be delivered, via a cannula 223, to a patient wearing the IDD 200. A pump 222 controllably delivers designated amounts of medication from the reservoir 221 through the cannula 223. The reservoir 221 may be filled via a septum or fill port 220 using a syringe. The IDD may also include a manual insertion mechanism (not shown) for inserting the cannula XX into a patient.

The pump 222 includes a piezoelectric actuator 100 according to an example embodiment described herein, in order to pull liquid medication from the reservoir 221 into a barrel, and to pump liquid medication from the barrel to a patient, e.g. via the cannula 223.

According to one or more example embodiments, an actuator as described herein may enable the removal/omission of components within an IDD, and may enable a reduction of the size of the IDD.

It may be understood that the example embodiments described herein may be considered in a descriptive sense only and not for purposes of limitation. Descriptions of features or aspects within each example embodiment may be considered as available for other similar features or aspects in other example embodiments.

While example embodiments have been described with reference to the figures, it will be understood by those of ordinary skill in the art that various changes in form and details may be made therein without departing from the spirit and scope as defined by the following claims.

What is claimed is:

1. A piezoelectric actuator comprising:
a plunger,
a frame attached to the plunger,
an advance piezoelectric unit disposed within the frame, the advance piezoelectric unit comprising a first piezoelectric element, wherein application of an electric field to the first piezoelectric element causes the first piezoelectric element to expand along a first axis, and
a lock piezoelectric unit disposed within the frame, the lock piezoelectric unit comprising a second piezoelectric element, wherein application of an electric field to the second piezoelectric element causes the second piezoelectric element to expand along a second axis, wherein the second axis forms a non-zero angle with respect to the first axis.

2. The piezoelectric actuator according to claim 1, wherein the second angle is orthogonal with respect to the first axis.

3. The piezoelectric actuator according to claim 2, wherein the plunger has an exterior circumference configured to fit within a barrel.

4. The piezoelectric actuator according to claim 2, wherein the first piezoelectric element comprises a plurality of first piezoelectric elements arranged in a stack.

5. The piezoelectric actuator according to claim 2, wherein the second piezoelectric element comprises a plurality of second piezoelectric elements arranged in a stack.

6. The piezoelectric actuator according to claim 4, wherein each of the plurality of first piezoelectric elements is circular in a cross-section normal to the first axis.

7. The piezoelectric actuator according to claim 5, wherein each of the plurality of second piezoelectric elements is circular in a cross-section normal to the first axis.

8. The piezoelectric actuator according to claim 7, wherein application of an electric field to the plurality of second piezoelectric element causes each of the plurality of second piezoelectric elements to expand in a radial direction thereof.

9. A fluid medication delivery method comprising:
holding a fluid medication within a barrel of a syringe, wherein the syringe comprises the barrel and a piezoelectric actuator within a barrel of a syringe, the piezoelectric actuator comprising a plunger, a frame attached to the plunger, an advance piezoelectric unit disposed within the frame and comprising a first piezoelectric element, and a lock piezoelectric unit disposed within the frame and comprising a second piezoelectric element;
applying a first electric field to the second piezoelectric element thereby causing the second piezoelectric element to expand in a radial direction of the barrel and exert a pressure on an inner circumference of the barrel, clamping the frame in a rear position in the barrel;
while maintaining the first electric field applied to the second piezoelectric element, applying a second electric field to the first piezoelectric element thereby causing the first piezoelectric element to extend along an axial direction of the barrel, orthogonal to the radial direction, thereby advancing the plunger toward a front end of the barrel and pumping fluid medication out of the syringe;
while maintaining the second electric field applied to the first piezoelectric element, removing the first electric field from the second piezoelectric element thereby causing the second piezoelectric element to retract in the radial direction away from the inner circumference of the barrel, releasing the clamping of the frame; and
releasing the second electric field from the first piezoelectric element thereby causing the first piezoelectric element to retract in the axial direction pulling the frame toward the plunger.

10. The method according to claim 9, further comprising repeating the holding, applying the first electric field; applying the second electric field; removing the first electric field; and removing the second electric field.

11. The method according to claim 9, wherein the first piezoelectric element comprises a plurality of first piezoelectric elements arranged in a stack.

12. The method according to claim 9, wherein the second piezoelectric element comprises a plurality of second piezoelectric elements arranged in a stack.

13. The method according to claim 11, wherein each of the plurality of first piezoelectric elements is circular in a cross-section normal to the axial direction of the barrel.

14. The method according to claim 12, wherein each of the plurality of second piezoelectric elements is circular in a cross-section normal to the axial direction of the barrel.

15. A fluid medication pump comprising:
a barrel, and
a piezoelectric actuator disposed within the barrel, the piezoelectric actuator comprising:
a plunger,
a frame attached to the plunger,
an advance piezoelectric unit disposed within the frame, the advance piezoelectric unit comprising a first piezoelectric element, wherein application of an electric field to the first piezoelectric element causes the first piezoelectric element to expand along a first axis, and
a lock piezoelectric unit disposed within the frame, the lock piezoelectric unit comprising a second piezoelectric element, wherein application of an electric field to the second piezoelectric element causes the second piezoelectric element to expand along a second axis, wherein the second axis forms a non-zero angle with respect to the first axis.

16. The piezoelectric actuator according to claim 15, wherein the second angle is orthogonal with respect to the first axis.

17. The fluid medication pump according to claim 16, wherein
the barrel comprises a cylinder extending along the first axis;
the plunger has an outer circumference configured to fit within an inner circumference of the barrel.

18. The fluid medication pump according to claim 16, wherein the first piezoelectric element comprises a plurality of first piezoelectric elements arranged in a stack.

19. The fluid medication pump according to claim 16, wherein the second piezoelectric element comprises a plurality of second piezoelectric elements arranged in a stack.

20. The fluid medication pump according to claim 18, wherein each of the plurality of first piezoelectric elements is circular in a cross-section normal to the first axis.

21. The fluid medication pump according to claim 19, wherein each of the plurality of second piezoelectric elements is circular in a cross-section normal to the first axis.

22. The fluid medication pump according to claim 21, wherein application of an electric field to the plurality of second piezoelectric elements causes each of the plurality of second piezoelectric elements to expand in a radial direction thereof.
